(19) United States
(12) Patent Application Publication
(10) Pub. No.: US 2007/0077204 A1
(43) Pub. Date: Apr. 5, 2007

(54) Olfactory identification tests for cognitive diseases and disorders

(75) Inventors: Davangere P. Devanand, Tarrytown, NY (US); Matthias H. Tabert, New York, NY (US); Xinhua Liu, Flushing, NY (US); Richard L. Doty, Haddon Heights, NJ (US)

Correspondence Address: WilmerHale/Columbia University, 399 PARK AVENUE, NEW YORK, NY, 10022, US

(73) Assignee: THE TRUSTEES OF COLUMBIA UNIVERSITY IN THE CITY OF NEW YORK, New York, NY (US)

(21) Appl. No.: 11/404,484

(22) Filed: Apr. 13, 2006

Related U.S. Application Data
(60) Provisional application No. 60/689,272, filed on Jun. 10, 2005.

(51) Int. Cl. A61K 49/00

(52) U.S. Cl. 424/9.2

(57) ABSTRACT

The present invention provides smell tests (odor identification tests) that are shorter that UPSIT, yet has a statistical sensitivity and specificity equivalent to or better than UPSIT. The odor identification tests of the invention are based on a core set of six odorants, where the six odorants can be selected from the following group of odorants: menthol, clove, leather, strawberry, lilac, pineapple, smoke, soap, natural gas and lemon. The present invention provides odor identification tests that can: (1) discriminate between subjects who are normal and who have a neuropsychiatric condition, cognitive disease or disorder, and/or (2) predict which subjects with mild cognitive disorders will develop various neuropsychiatric conditions or cognitive diseases and disorders. In one embodiment, the test and methods of the invention can provide an early prediction or diagnosis of Alzheimer's disease that is important for patients (including patients who have mild cognitive disorders, such as MCI) and clinicians to make plans for the future and to institute early treatment.

The invention claims priority to U.S. Ser. No. 60/689,272, filed Jun. 10, 2005.

The invention disclosed herein was made in part with U.S. Government support from the National Institutes on Aging grant numbers AG17761 and 1K01AG21548

This disclosure contains material that is subject to copyright protection. The copyright owner has no objection to the facsimile reproduction by anyone of the patent document or the patent disclosure, as it appears in the U.S. Patent and Trademark Office patent file or records, but otherwise reserves any and all copyright rights.

All patent applications, published patent applications, issued and granted patents, texts, and literature references cited in this specification are hereby incorporated herein by reference in their entirety.

### BACKGROUND OF THE INVENTION

Impaired odor identification has been implicated in a number of neuropsychiatric conditions or cognitive diseases and disorders, including Alzheimer's disease, Parkinson's disease, Huntington's disease, Korsakoff's amnestic syndrome, acquired immunodeficiency syndrome (AIDS), amyotrophic lateral sclerosis, motor neuron disease, schizophrenia, and advanced anorexia.

The University of Pennsylvania Smell Identification Test (UPSIT) is an odor identification test that measures smell function in nonlaboratory settings without the use of complex olfactometric equipment or cumbersome sniff bottles. The UPSIT test is a 40-item “scratch and sniff” microencapsulated odorant test, that is commercially known as the Smell Identification Test™ (Sensonics, Inc., Haddonfield, N.J.). Briefly, this test consists of four envelope-sized booklets, each containing 10 scratch and sniff odorants. The odorants are embedded in 10- to 50-μm urea-formaldehyde polymer microcapsules fixed in a binder and positioned on brown strips at the bottom of the pages of test booklets. The stimuli are released by the scratching of the strips with a pencil tip. Above each odorant strip is a multiple-choice question with four alternative responses for each item. For example, one of the items reads: “This odor smells must like: a) chocolate; b) banana; c) onion; or d) fruit punch.” The test is forced-choice, which means that the subject is required to mark one of the four alternatives even if no smell is perceived.

Although the UPSIT test is used in research to assess odor identification deficits, it is less widely used in clinical practice, in part because administration takes 15-25 minutes. Therefore, a subset of the odors from the UPSIT test was used to fashion two shorter tests: the Cross-Cultural Smell Identification Test (CC-SIT, now known as The Brief Smell Identification Test™ (B-SIT), Sensonics, Inc.) and the Pocket Smell Test (PST, Sensonics, Inc.). The B-SIT consists of twelve odors, and is based on a forced multiple choice from a list of four items (see U.S. Patent Application Publication No. US 2002/0139170). The PST consists of three odors, and provides a very brief screen of gross olfactory dysfunction. Although the B-SIT and PST tests reduce the time of the UPSIT test, neither provides an equivalent statistical sensitivity for predicting, identifying or discriminating cognitive disorders as compared to UPSIT.

### SUMMARY OF THE INVENTION

The present invention provides odor identification tests that can (1) discriminate between subjects who are normal and who have a neuropsychiatric condition, cognitive disease or disorder, and/or (2) predict which subjects with mild cognitive disorders, including mild cognitive impairment (MCI), will develop various neuropsychiatric conditions or cognitive diseases and disorders.

In one aspect, the invention provides an odor identification test, the test comprising from six (6) odorants to about twenty (20) odorants, wherein the at least six odorants comprise at least six odorants selected from the group consisting of: menthol, clove, leather, strawberry, lilac, pineapple, smoke, soap, natural gas and lemon. In addition to this ‘core set’ of six odorants, the tests of this invention can further comprise additional odorants, from about one additional odorant to about fourteen additional odorants. The additional odorants can be selected, for example, from the group consisting of: menthol, clove, leather, strawberry, lilac, pineapple, smoke, soap, natural gas, lemon, pizza, cherry, motor oil, mint, banana, onion, licorice, gasoline, gingerbread, root beer, wintergreen, watermelon, paint thinner, pine, grape, rose, peanut, bubble gum, dill pickle, chocolate, peach, turpentine, cedar, coconut, cinnamon, cheddar cheese, lime, grass, orange and fruit punch. Odorants that are selected in addition to the core set of six odorants should not be the same as any of the six odorants that are selected to be core odorants. In one aspect, an odor identification test does not comprise any one of the following odorants: cheddar cheese, lime, grass, orange and fruit punch.

Thus, in one aspect, the invention provides an odor-identification test for determining whether a subject has a neuropsychiatric condition, cognitive disease or disorder, or a risk for developing a neuropsychiatric condition, cognitive disease or disorder, the test comprising from six to twenty different odorants, wherein at least six odorants are selected from the group consisting of: menthol, clove, leather, strawberry, lilac, pineapple, smoke, soap, natural gas, and lemon.

In another aspect, the invention provides an odor-identification test for determining whether a subject has a neuropsychiatric condition, cognitive disease or disorder, or a risk for developing a neuropsychiatric condition, cognitive disease or disorder, the test comprising from seven to twenty different odorants, wherein at least six odorants are selected from the group consisting of: menthol, clove, leather, strawberry, lilac, pineapple, smoke, soap, natural gas and lemon, and wherein the additional odorants are selected from the group consisting of: menthol, clove, leather, strawberry, lilac, pineapple, smoke, soap, natural gas, lemon, pizza, cherry, motor oil, mint, banana, onion, licorice, gasoline, gingerbread, root beer, wintergreen, watermelon, paint thinner, pine, grape, rose, peanut, bubble gum, dill pickle, chocolate, peach, turpentine, cedar, coconut, cinnamon, cheddar cheese, lime, grass, orange, and fruit punch.

In another aspect, the invention provides an odor-identification test for determining whether a subject has a neuropsychiatric condition, cognitive disease or disorder, or a risk for developing a neuropsychiatric condition, cognitive disease or disorder, the test comprising from seven to twenty different odorants, wherein at least six odorants are selected from the group consisting of: menthol, clove, leather, strawberry, lilac, pineapple, smoke, soap, natural gas and lemon, and wherein the additional odorants are selected from the group consisting of: menthol, clove, leather, strawberry, lilac, pineapple, smoke, soap, natural gas, lemon, pizza, cherry, motor oil, mint, banana, onion, licorice, gasoline, gingerbread, root beer, wintergreen, watermelon, paint thinner, pine, grape, rose, peanut, bubble gum, dill pickle, chocolate, peach, turpentine, cedar, coconut, and cinnamon.

In one aspect, the invention provides an odor-identification test for determining whether a subject has a neuropsychiatric condition, cognitive disease or disorder, or a risk for developing a neuropsychiatric condition, cognitive disease or disorder, the test comprising exactly seven different odorants, wherein the seven odorants are selected from the group consisting of menthol, clove, leather, strawberry, lilac, pineapple, smoke, soap, natural gas, and lemon.

In one aspect, the invention provides an odor-identification test for determining whether a subject has a neuropsychiatric condition, cognitive disease or disorder, or a risk for developing a neuropsychiatric condition, cognitive disease or disorder, the test comprising exactly eight different odorants, wherein the eight odorants are selected from the group consisting of menthol, clove, leather, strawberry, lilac, pineapple, smoke, soap, natural gas, and lemon.

In one aspect, the invention provides an odor-identification test for determining whether a subject has a neuropsychiatric condition, cognitive disease or disorder, or a risk for developing a neuropsychiatric condition, cognitive disease or disorder, the test comprising exactly nine different odorants, wherein the nine odorants are selected from the group consisting of menthol, clove, leather, strawberry, lilac, pineapple, smoke, soap, natural gas, and lemon.

In one aspect, the invention provides an odor-identification test for determining whether a subject has a neuropsychiatric condition, cognitive disease or disorder, or a risk for developing a neuropsychiatric condition, cognitive disease or disorder, the test comprising exactly ten different odorants, wherein the ten odorants are menthol, clove, leather, strawberry, lilac, pineapple, smoke, soap, natural gas, and lemon. In another aspect, the invention provides an odor-identification test for determining whether a subject has a neuropsychiatric condition, cognitive disease or disorder, or a risk for developing a neuropsychiatric condition, cognitive disease or disorder, the test consisting essentially of ten different odorants, wherein the ten odorants are menthol, clove, leather, strawberry, lilac, pineapple, smoke, soap, natural gas, and lemon.

In the odor-identification tests of the invention, the odorants can be contained in microcapsules, an absorbent substance, or in a bottle. The odorants can be in liquid or solid form. When the odorants are contained in microcapsules, the microcapsules can be placed or embedded in a substrate, such as a strip. The strip can be a “scratch-and-sniff” strip, where scratching the strip causes a portion of the microcapsules to break open and release the odorants. For example, a test-taker can scratch the strip with the point of a sharpened pencil or some other pointed device and then smell the strip.

In the odor-identification tests of the invention, the tests can further comprise a multiple-choice question matched to each odorant, wherein each multiple-choice question asks a test-taker to identify the name of the matched odorant from a set of odorant names. The multiple-choice question can provide a set of odorant names that ranges from 2 names to 10 names, for example. In one aspect, the multiple-choice question provides a set of four odorant names (for example, see Table 1). Alternatively, the tests can further comprise a non-forced answer question, where the question simply asks the test-taker to identify the odorant without providing the test-taker a set of potential answers to choose from. In this alternative, the test-taker can write down his or her answer or the test-taker can inform an administrator of the test of the answer.

In one aspect, the tests can further comprise a booklet, wherein the booklet can comprise a different page for each odorant, wherein each page can comprise: (a) a strip or other substrate comprising microcapsules comprising an odorant, and (b) a multiple-choice question asking a test-taker to identify the name of the odorant (that is released from the strip or other substrate upon scratching or rubbing the strip or other substrate).

In another aspect, the tests of the invention have a sensitivity and a specificity greater than 80% with respect to a test-taker's score, wherein the score is 80% of the odorants correctly identified. Thus, in one aspect, the invention provides invention provides an odor-identification test for determining whether a subject has a neuropsychiatric condition, cognitive disease or disorder, or a risk for developing a neuropsychiatric condition, cognitive disease or disorder, the test comprising from six to twenty different odorants, wherein at least six odorants are selected from the group consisting of: menthol, clove, leather, strawberry, lilac, pineapple, smoke, soap, natural gas, and lemon, and wherein the test comprises a sensitivity and specificity greater than 80% for a test-taker score of 80% of odorants correctly identified.

In all of the aspects of the invention, including tests, kits and methods, a neuropsychiatric condition, cognitive disease or disorder can comprise Alzheimer's disease (AD), Parkinson's disease, Huntington's disease, Korsakoff's amnestic syndrome, acquired immunodeficiency syndrome (AIDS), amyotrophic lateral sclerosis, motor neuron disease, schizophrenia, advanced anorexia, frontotemporal dementia, lewy body dementia, vascular dementia, or any combination thereof.

In one aspect, the invention provides a method for discriminating whether a subject is normal or suffers from a neuropsychiatric condition, cognitive disease or disorder, the method comprising: (a) administering to the subject an odor-identification test of the invention; (b) scoring the number of odorants correctly identified by the subject; and (c) analyzing the score to diagnose the subject as normal or as having a neuropsychiatric condition, cognitive disease or disorder. With respect to the analyzing step, the subject can be diagnosed as normal if the subject correctly identifies at least 70%, 80% or 90% of the odorants in the test. In another aspect with respect to the analyzing step, the subject can be diagnosed as normal if (i) the subject correctly identifies at least 70%, 80% or 90% of the odorants; and (ii) the odor-identification test has a sensitivity and specificity greater than 80% with respect to the score of the number of correct odorants identified. In another aspect, the method can further comprise conducting a clinical test, the clinical test comprising a neuropsychological test of memory or other cognitive abilities, a test of ability to perform daily functional activities, a brain imaging test or a biomarker test. The brain imaging test can comprise, for example, a magnetic resonance imaging (MRI) test, a single photon emission computerized tomography (SPECT) test, or a positron emission tomography (PET) test. The biomarker test can comprise, for example, a blood biomarker test or a cerebrospinal fluid biomarker test.

In another aspect, the invention provides a method for predicting whether a subject who suffers from a mild cognitive disorder will develop a more severe neuropsychiatric condition, cognitive disease or disorder, the method comprising: (a) administering to the subject an odor-identification test of the invention; (b) scoring the number of odorants correctly identified by the subject; and (c) analyzing the score to predict whether the subject will develop a more severe neuropsychiatric condition, cognitive disease or disorder. With respect to the analyzing step, the analysis can comprise a diagnosis where the subject is not predicted to develop a more severe neuropsychiatric condition, cognitive disease or disorder if the subject correctly identified at least 70%, 80% or 90% of the odorants. In another aspect with respect to the analyzing step, the subject is not predicted to develop a more severe neuropsychiatric condition, cognitive disease or disorder if: (i) the subject correctly identified at least 70%, 80% or 90% of the odorants; and (ii) the odor-identification test has a sensitivity and specificity greater than 80% with respect to the score of the number of correct odorants identified. In one aspect, the mild cognitive disorder is Mild Cognitive Impairment and the more severe neuropsychiatric condition, cognitive disease or disorder is Alzheimer's Disease. In another aspect, the method can further comprise conducting a clinical test, the clinical test comprising a neuropsychological test of memory or other cognitive abilities, a test of ability to perform daily functional activities, a brain imaging test or a biomarker test. The brain imaging test can comprise, for example, a magnetic resonance imaging (MRI) test, a single photon emission computerized tomography (SPECT) test, or a positron emission tomography (PET) test. The biomarker test can comprise, for example, a blood biomarker test or a cerebrospinal fluid biomarker test.

With respect to the methods for predicting whether a subject who suffers from a mild cognitive disorder will develop a neuropsychiatric condition, cognitive disease or disorder, the mild cognitive disorder can comprise, for example, Mild Cognitive Impairment (MCI), age associated memory impairment (AAMI), age related cognitive decline (ARCD), Benign Senescent Forgetfulness (BSF), or Cognitive Impairment No Dementia (CIND). Again, the neuropsychiatric condition, cognitive disease or disorder can comprise Alzheimer's disease, Parkinson's disease, Huntington's disease, Korsakoff's amnestic syndrome, acquired immunodeficiency syndrome (AIDS), amyotrophic lateral sclerosis, motor neuron disease, schizophrenia, advanced anorexia, or any combination thereof. In one aspect, the mild cognitive disorder is MCI and the neuropsychiatric condition, cognitive disease or disorder is Alzheimer's disease.

In one aspect, the invention provides an odor-identification test kit for determining whether a subject has Alzheimer's disease, or a risk for developing Alzheimer's disease, the odor-identification test kit comprising: (a) an odor-identification test of the invention; and (b) instructions for taking the test. In the kit, the odorants can be contained in microcapsules, wherein the microcapsules for each odorant are embedded or place in a strip (or other substrate), wherein there is a different strip for the microcapsules for each odorant. Also, the test of the kit can further comprise a multiple-choice question matched to each strip, and wherein the instructions comprise: (a) an instruction to a test-taker to scratch the strip with a pencil or another pointed device; (b) an instruction to the test-taker to smell the scratched strip; and (c) an instruction to the test-taker to answer the multiple-choice question matched to the strip, wherein the multiple-choice question asks the test-taker to identify the name of the odorant from the scratched strip, and wherein the multiple-choice question provides a set of potential answers comprising different odorant names. The odor-identification test kit can further comprise an answer-key. In a variation, the kit can comprise strips comprising odorants, where the strips are individually numbered to correspond to multiple-choice questions, where the multiple-choice questions are presented to a test-taker online. In this variation, the kit should further comprise an instruction sheet directing the test-taker or test-administrator to an internet address of a website. The website can provide the ability for the test-taker to mark his or her answer selections on the website. Additionally, the kit can provide a CD-ROM or other electronic storage device that contains instructions, multiple-choice questions and an answer key that can be printed onto paper by the test-taker or test-administrator.

### DETAILED DESCRIPTION OF THE INVENTION

The present invention has succeeded in providing a smell test that is shorter that UPSIT, yet has a statistical sensitivity and specificity for predicting risk of Alzheimer's disease equivalent to or better than UPSIT. The odor identification tests of the invention are based on a core set of six odorants, where the six odorants can be selected from the following group of odorants: menthol, clove, leather, strawberry, lilac, pineapple, smoke, soap, natural gas and lemon. In addition to the core set of six odorants, the present tests can further comprise additional odorants, from an additional one odorant to an additional fourteen odorants. In one embodiment, the additional odorants can be selected, for example, from the following group of odorants: menthol, clove, leather, strawberry, lilac, pineapple, smoke, soap, natural gas, lemon, pizza, cherry, motor oil, mint, banana, onion, licorice, gasoline, gingerbread, root beer, wintergreen, watermelon, paint thinner, pine, grape, rose, peanut, bubble gum, dill pickle, chocolate, peach, turpentine, cedar, coconut, cinnamon, cheddar cheese, lime, grass, orange and fruit punch. This list for additional odorants is not meant to be limiting, and one skilled in the art can choose essentially any odorant to be an additional odorant. In one embodiment, the odor identification test does not comprise any one of the following odorants: cheddar cheese, lime, grass, orange and fruit punch. Therefore, the invention contemplates tests that contain six odorants to twenty odorants, where each test contains at least six odorants selected from the core odorant group of menthol, clove, leather, strawberry, lilac, pineapple, smoke, soap, natural gas and lemon.

In one embodiment, the test and methods of the invention can provide an early prediction or diagnosis of Alzheimer's disease that is important for patients and clinicians to make plans for the future and to institute early treatment.

In another embodiment, the simplicity and short duration of the administration of the invention's tests make it suitable for use by physicians in their offices when seeing patients.

As used herein, the term “odorant,” refers to a substance that has or emits a smell, including a substance that is used to impart a particular scent or odor to a product. Therefore, an individual odorant herein can comprise a single molecular entity (or chemical compound) or a combination of different molecular entities. A single molecular entity or a combination of different molecular entities, as a single substance, can give rise to a particular odor, fragrance or smell. The molecular entity or entities that comprise an odorant can be natural or synthetic, as long as the odors from the odorants can be identified and named by normal subjects as the designated odor in question.

As used herein, the term “sensitivity” of an odor-identification test refers to the proportion of subjects having a neuropsychiatric condition, cognitive disease or disorder, correctly identified by the test as having the condition, disease, or disorder (also called “true positives”).

As used herein, the term “specificity” of an odor-identification test refers to the proportion of subjects not having a neuropsychiatric condition, cognitive disease or disorder, correctly identified by the test as not having the condition, cognitive disease or disorder (also called “true negatives”).

For both sensitivity and specificity, the values fluctuate depending on which cutoff score, i.e., the number of correct responses used for a test (for example, see Table 4) is used for a test.

The tests can be in a variety of formats. In one embodiment, the odorants can be presented to a test-taker in form of microcapsules that are fixed on a strip or some other substrate. The UPSIT and B-SIT, for example, present their odorants by using 10-50 μm microcapsules fixed on strips that are presented in booklet pages. The strips can be scratched with a pencil that releases the odorants from the microcapsules, i.e, “scratch and sniff.” The tests of the invention can also comprise odorants that are encapsulated in such a manner. In another embodiment, the odorants can be presented by smell-bottles, where the odorants can be in solid or liquid form in the bottle. The test-taker opens the smell-bottle by removing a cap, lid, cork, bottle-cap or some other device that keeps the odorants in the bottle, and attempts to identify the odor. In another embodiment, the odorants can be presented by smell-pens or smell-sticks, for example, see “Sniffin' Sticks,” Hummel, T. et al., Chem. Senses, 1997, 22:39-52. Sniffin' sticks are felt-tip pens that contain a tampon that is filled with dissolved odorants. For odor presentation, the cap of the pen is removed by the test-taker and the pen's tip is placed in front of the test-taker's nostrils. The odorants can be presented by other devices known to one skilled in the art.

In one embodiment, the odorants that are presented to the test-taker can be in the context of a multiple-choice question test. After the odorant is presented to the test-taker, for example, by rupture of microcapsules (i.e., scratch and sniff), smell-bottles or smell-sticks/pens, then the test-taker must attempt to identify the name of the odorant by selecting an answer from a set of answers. This format is known in the art as “forced answer” or “forced multiple choice,” because the test-taker is forced to provide an answer. In forced choice tests a subject is asked to identify the name of a smelled odor given two or more possible odor names to choose from. Forced-choice tests can be less susceptible than non-forced choice tests to contamination by response biases (i.e., the conservatism or liberalism in reporting the presence of an odor under uncertain conditions) and therefore can be more reliable and sensitive.

In one embodiment, answer selections for multiple-choice questions can be selected from the following list of odors (which is not meant to be limiting): gasoline, pizza, peanuts, lilac, dill pickle, bubble gum, wintergreen, watermelon, tomato, licorice, strawberry, menthol, whiskey, honey, lime, cherry, grass, motor oil, pineapple, skunk, mint, fruit punch, cola, banana, garlic, cherry, clove, chili, leather, apple, coconut, cedar, chocolate, onion, soap, pumpkin pie, cheddar cheese, paint thinner, cinnamon, pine, rose, lemon, peach, dill pickle, root beer, black pepper, gingerbread, turpentine, smoke, musk, lime, peach, orange, grape, grass, rose, bubble gum, natural gas, and peanut. The list of odors can involve essentially any odor. A multiple-choice question can comprise at least the names of two odors, wherein the name for one odor is the correct answer or odor name matched to an odorant of a test. In one embodiment, multiple-choice questions can be selected from those presented in Table 1 below

TABLE 1
Exemplary Multiple-Choice Questions
Multiple-Choice Question

This odor smells most like: (a) gasoline, (b) pizza, (c) peanuts, or (d) lilac
This odor smells most like: (a) dill pickle, (b) bubble gum, (c) wintergreen, or (d) watermelon
This odor smells most like: (a) tomato, (b) licorice, (c) strawberry, or (d) menthol
This odor smells most like: (a) whiskey, (b) honey, (c) lime, or (d) cherry
This odor smells most like: (a) grass, (b) pizza, (c) motor oil, or (d) pineapple
This odor smells most like: (a) skunk, (b) mint, (c) fruit punch, or (d) cola
This odor smells most like: (a) banana, (b) garlic, (c) cherry, or (d) motor-oil
This odor smells most like: (a) licorice, (b) clove, (c) chili, or (d) banana
This odor smells most like: (a) clove, (b) lilac, (c) leather, or (d) apple
This odor smells most like: (a) skunk, (b) coconut, (c) cedar, or (d) honey
This odor smells most like: (a) chocolate, (b) banana, (c) onion, or (d) fruit punch
This odor smells most like: (a) soap, (b) fruit punch, (c) menthol, or (d) pumpkin pie
This odor smells most like: (a) licorice, (b) pineapple, (c) cheddar cheese, or (d) cherry
This odor smells most like: (a) paint thinner, (b) cherry, (c) coconut, or (d) cheddar cheese
This odor smells most like: (a) cola, (b) cinnamon, (c) pine, or (d) coconut
This odor smells most like: (a) rose, (b) lemon, (c) peach, or (d) gasoline
This odor smells most like: (a) strawberry, (b) dill pickle, (c) chocolate, or (d) cedar
This odor smells most like: (a) cedar, (b) gasoline, (c) lemon, or (d) root beer
This odor smells most like: (a) lemon, (b) chocolate, (c) root beer, or (d) black pepper
This odor smells most like: (a) menthol, (b) apple, (c) gingerbread, or (d) cheddar cheese
This odor smells most like: (a) lilac, (b) chili, (c) coconut, or (d) whiskey
This odor smells most like: (a) turpentine, (b) soap, (c) fruit punch, or (d) cola
This odor smells most like: (a) chocolate, (b) peach, (c) leather, or (d) pizza
This odor smells most like: (a) root beer, (b) watermelon, (c) banana, or (d) smoke
This odor smells most like: (a) pineapple, (b) dill pickle, (c) root beer, or (d) black pepper
This odor smells most like: (a) smoke, (b) whiskey, (c) pineapple, or (d) onion
This odor smells most like: (a) musk, (b) garlic, (c) turpentine, or (d) lime
This odor smells most like: (a) cheddar cheese, (b) orange, (c) bubble gum, or (d)
This odor smells most like: (a) lime, (b) wintergreen, (c) pumpkin pie, or (d) leather
This odor smells most like: (a) chili, (b) menthol, (c) orange, or (d) watermelon
This odor smells most like: (a) watermelon, (b) peanut, (c) rose, or (d) paint thinner
This odor smells most like: (a) mint, (b) gingerbread, (c) grass, or (d) strawberry
This odor smells most like: (a) dill pickle, (b) grass, (c) smoke, or (d) peach
This odor smells most like: (a) pine, (b) smoke, (c) lilac, or (d) orange
This odor smells most like: (a) pizza, (b) turpentine, (c) clove, or (d) grape
This odor smells most like: (a) motor oil, (b) pumpkin pie, (c) rose, or (d) lemon
This odor smells most like: (a) soap, (b) black pepper, (c) licorice, or (d) peanut
This odor smells most like: (a) orange, (b) musk, (c) cola, or (d) natural gas
This odor smells most like: (a) lime, (b) rose, (c) mint, or (d) bubble gum
This odor smells most like: (a) peanut, (b) lemon, (c) apple, or (d) root beer

It is understood to one skilled in the art that the answer items in each multiple-question above can be varied. It is also understood to one skilled in the art that the tests of the invention can be presented in any language, where questions are adapted to the language of the country in which the test is conducted. If the language of the country does not possess a word for an odorant to be tested, then a different odorant can be used, as long as the different odorant is within the guidelines of the invention as disclosed herein. If the language of the country does not have translations for at least six of the ten core odorants, i.e, menthol, clove, leather, strawberry, lilac, pineapple, smoke, soap, natural gas and lemon, then the English name of these odorants can be used.

The tests of the invention can be used, for example, in any country that grows, sells or uses at least six products or things that emit or have one of the following core odors: menthol, clove, leather, strawberry, lilac, pineapple, smoke, soap, natural gas and lemon.

In one embodiment, the tests of the invention can comprise a booklet or booklets, wherein individual pages of the booklet each has (a) a strip containing microcapsules of an odorant, and (b) a multiple choice question asking whether the odorant (or odor) that is released from rupture of the microcapsules on the strip smells like (or most like) one of a set of odor names. For example, a page of the booklet can contain a strip containing microcapsules of the pizza odorant. The test taker scratches the strip with some device, such as a pencil, to release the odorant molecules from the microcapsules. The test taker then smells the scratched strip and reads the multiple-choice question on the page. The multiple choice question asks the test-taker to try to identify the name of the odor smelled from the strip, where the question provides a set of different odor answers that includes the correct answer, in this case, pizza. For example, the question can state: “This odor smells most like: (a) gasoline, (b) pizza, (c) peanuts or (d) lilac.” In another embodiment, each page of a booklet can comprise more than one odorant strip and corresponding multiple-choice question. In another embodiment, the tests of the invention can present odorants and questions in the form of a rotating test panel (see U.S. Pat. No. 6,557,394).

In another embodiment, odor identification kits are provided that comprise an odor-identification test or tests of the invention. The kit can further contain instructions for completing the test. The instructions can be part of a booklet that contains the odorant-strips and questions, or the instructions can be separate. If the kit contains an odor-identification test that comprises smell-sticks or smell-bottles, then the instructions can be supplied separately. The kit can further comprise an answer key, so that a test-taker or test-administrator can determine the correct identity for each odorant. Exemplary test booklets and kits can be similar in format and appearance to the booklets and kits described in International Patent Publication WO 03/051177.

In one embodiment, the tests of the invention can be administered to a subject by a physician. In another embodiment, the tests of the invention can be self-administered by a subject to himself or herself.

In one embodiment, the invention provides methods for determining whether a subject may have a neuropsychiatric condition, cognitive disease or disorder in a subject by testing the subject with an odor identification test of the invention. In other words, the tests of the invention can be used to discriminate between subjects who are normal and subjects who have a neuropsychiatric condition, cognitive disease or disorder. A diagnosis relating to whether a subject is normal or has a neuropsychiatric condition, cognitive disease or disorder, can be a preliminary diagnosis. A diagnosis on the condition of a subject made from using a test of the invention can have varying degrees of certainty, such that the test of the invention can involve assessing or determining the probability that a subject actually has a neuropsychiatric condition, cognitive disease or disorder. Therefore, tests of the invention can be used in conjunction with other clinical tests in order to provide a more definitive diagnosis or analysis on a subject's condition. Exemplary clinical tests include, but are not limited to, neuropsychological tests of memory and other cognitive abilities, tests of ability to perform daily functional activities, brain imaging tests (including MRI (magnetic resonance imaging), SPECT (single photon emission computerized tomography), and PET (positron emission tomography)), and tests of biomarkers in blood, cerebrospinal fluid and other bodily fluids and tissues.

In another embodiment, the invention provides methods for predicting whether a subject will come to have a neuropsychiatric condition, cognitive disease or disorder by testing the subject with an odor identification test of the invention. With respect to prediction, the invention refers to assessing or determining the probability or relative risk that a subject has for developing a neuropsychiatric condition, cognitive disease or disorder. In one embodiment, the tests of the invention can be used to predict whether a subject who has a mild cognitive disorder is likely to develop a more severe neuropsychiatric condition, cognitive disease or disorder. In one embodiment, the mild cognitive disorder can comprise, for example, Mild Cognitive Impairment (MCI) (which herein, includes cognitive impairments ranging from minimal to mild), mild memory loss, age associated memory impairment (AAMI), age related cognitive decline (ARCD), Benign Senescent Forgetfulness (BSF), or Cognitive Impairment No Dementia (CIND). A mild cognitive disorder includes disorders that require cognitive impairment as a clinical feature of the syndrome and subjects do not meet diagnostic criteria for dementia, e.g., DSM-IV TR criteria for dementia. Among these disorders, mild cognitive impairment is a condition characterized by cognitive, most commonly memory, deficits in the absence of clinically significant functional impairment. In one embodiment, the invention provides methods for predicting whether a subject who has MCI is likely to develop Alzheimer's disease. With respect to assessing or determining the probability or relative risk that a subject has for developing a neuropsychiatric condition, cognitive disease or disorder, the methods can further comprise or can be used in conjunction with additional clinical tests such as neuropsychological tests of memory and other cognitive abilities, tests of ability to perform daily functional activities, brain imaging tests (including MRI (magnetic resonance imaging), SPECT (single photon emission computerized tomography), and PET (positron emission tomography)), and tests of biomarkers in blood, cerebrospinal fluid and other bodily fluids and tissues.

The neuropsychiatric condition, cognitive disease or disorder referred to by the methods and compositions of the invention, include, but are not limited to, Alzheimer's disease, Parkinson's disease, Huntington's disease, Korsakoff's amnestic syndrome, acquired immunodeficiency syndrome (AIDS), amyotrophic lateral sclerosis, motor neuron disease, schizophrenia, advanced anorexia, frontotemporal dementia, lewy body dementia, and vascular dementia.

Additionally, the tests can be used in methods for predicting whether a subject will come to have a neuropsychiatric condition, cognitive disease or disorder by testing the subject with an odor identification test of the invention. In one embodiment, the subject presently has a mild-cognitive disorder. Subjects can be considered to be normal or can be considered not to be at risk for developing a more severe cognitive disorder, neuropsychiatric condition, or cognitive disease, if they correctly identify at least 70%, 80% or 90% of the odorants. Subjects who do not correctly identify at least 70%, 80% or 90% of the odorants, can be diagnosed or preliminarily diagnosed as having a risk for developing a neuropsychiatric condition, cognitive disease or disorder. Subjects who are diagnosed or determined to have a risk for developing a condition, disease or disorder can be subjected to additional clinical tests, as mentioned above, to confirm the diagnosis.

The invention provides for an odor-identification test for determining whether a subject has a neuropsychiatric condition, cognitive disease or disorder, or has a risk for developing a neuropsychiatric condition, cognitive disease or disorder, the test consisting of about 10 items. The invention provides for an odor-identification test for determining whether a subject has a neuropsychiatric condition, cognitive disease or disorder, or has a risk for developing a neuropsychiatric condition, cognitive disease or disorder, wherein the items have an intense odor, and comprising from at least six to about twenty different odorants, wherein the at least six odorants are selected from the group consisting of: lemon, paint thinner, dill pickle, smoke, onion, leather, turpentine, gasoline, peanut, and coconut.

### EXAMPLES

The following examples are representative of techniques employed by the inventors in carrying out aspects of the present invention. It should be appreciated that while these techniques are exemplary for the practice of the invention, those of skill in the art, in light of the present disclosure, will recognize that numerous modifications can be made without departing from the spirit and intended scope of the invention. Thus, the examples described below are provided to illustrate the present invention and are not included for the purpose of limiting the invention.

### Example 1

### A 10-Item Smell Identification Scale Related to Risk of Alzheimer's Disease

University of Pennsylvania Smell Identification Test (UPSIT) data from controls (n=63), mild cognitive impairment (MCI; n=147) and Alzheimer disease (AD) patients (n=100) were analyzed to derive an optimal subset of items related to risk of AD (i.e., healthy through MCI to early and moderate disease stages). The derived 10-item scale performed comparably to the UPSIT in classifying subjects, and strongly predicted conversion to AD on follow-up evaluation in MCI patients.

Early in the course of AD, neurofibrillary tangles appear in olfactory-related brain regions (e.g., anterior olfactory nucleus and entorhinal cortex). Olfactory deficits, which have been consistently observed in AD, occur early, are predictive of a future diagnosis of AD, and increase with disease severity. The UPSIT is widely used in research to assess odor identification deficits, but is less widely used in clinical practice, in part because administration takes 10-20 minutes. The Brief Smell Identification Test (B-SIT), consisting of 12 specific UPSIT items is a shorter test than UPSIT, but does not possess the same predictive and discrimination abilities as UPSIT. The current study provides a test that is as brief as B-SIT, but provides greater predictive and discriminatory abilities than B-SIT.

The methods described in this Example are used to determine which subsets of odors from UPSIT can comprise tests for the prediction or discrimination of neuropsychiatric conditions or cognitive diseases and disorders, including Alzheimer's disease, Parkinson's disease, Huntington's disease, Korsakoff's amnestic syndrome, acquired immunodeficiency syndrome (AIDS), amyotrophic lateral sclerosis, motor neuron disease, schizophrenia, and advanced anorexia. The methods described in this Example are used to design odor identification tests that can be conducted rapidly (less than 10 minutes) and have a greater statistical ability than B-SIT (or about the same statistical ability than UPSIT) for predicting or discriminating neuropsychiatric conditions or cognitive diseases and disorders.

Methods

Data from 310 subjects participating in separate studies at 3 medical centers were analyzed. MCI patients (n=147) and healthy controls (n=63) were recruited at the New York State Psychiatric Institute and Columbia University Medical Center. AD patients participated at Mount Sinai Medical Center (n=56) or in a study jointly performed at Jefferson Medical College and the Smell and Taste Center of the University of Pennsylvania (n=44). Detailed inclusion/exclusion criteria for the respective studies have been described (Devanand, D. P. et al., Am J Psychiatry, 2000, 157:1399-1405; Tabert, M. H. et al., Neurology, 2002, 58:758-764; Doty, R. L. et al., Brain Res Bull., 1987, 18:597-600; Serby, M. et al., Am J Psychiatry, 1991, 148:357-360).

Controls were recruited primarily by advertisement and followed annually. MCI patients presented with memory complaints to a Memory Disorders Center and were followed at 6-month intervals in a longitudinal study of early diagnostic markers of AD. Expert clinical raters, blind to UPSIT scores, made a consensus research diagnosis at each visit based on DSM-IV/NINCDS-ADRDA criteria (McKhann, G. et al., Neurology, 1984, 34:939-944). AD patients met criteria for a clinical diagnosis of probable AD based on NINCDS-ADRDA criteria (id.), had mild to moderate severity of illness, and had no major acute medical complications.

Statistical Analysis

Initially, 290 of the 310 subjects were ranked according to risk of AD: 1) “healthy” controls (n=63); 2) “low risk” MCI patients, followed for at least 2 years, who had not converted to AD within that period (n=96); 3) “high risk” MCI patients who converted to AD within 2 years of follow-up (n=31); and 4) AD patients (n=100). Cochran-Armitage linear trend tests were applied to UPSIT data with the goal of excluding the items that did not show a decreasing trend in the proportion of correct smell identifications across these ordinal groups (multiple test adjusted p-value above 0.20).

The remaining items were submitted to logistic regression (LR) analysis using a stepwise selection procedure. Dichotomous predictors (i.e., correct vs. incorrect on each UPSIT item) and a binary outcome variable (i.e., controls plus MCI non-converters at 2 years vs. MCI converters within 2 years plus AD patients) were used. Only items that were related [p<0.10] to the outcome after adjusting for all other items in the model were retained. A second logistic regression (LR) analyses using a likelihood score based selection procedure (Furnival, G. M. et al., Technometrics, 1974, 16:499-511) was also applied for statistical validation. Item subsets ranging in size from one-item to a set containing all items, and Akaike Information Criterion (AIC) was used to select the final set of items related to risk of AD. To further evaluate variability in item selection, the same procedures used to derive the 10-item scale (Cochran-Armitage tests and stepwise LR) were applied to 100 bootstrapping samples obtained by random cluster sampling with replacement from the original sample (n=290), where data from a subject is considered in a cluster.

The ability of the 10-item scale (see Table 3, the bolded odorants comprise the 10-item scale or test in this Example) in classifying subjects was compared to that of the UPSIT and B-SIT by calculating Receiver Operating Characteristic (ROC) curves for each scale. A non-parametric test (DeLong, E. R. et al., Biometrics, 1988, 44:837-845) was used to assess the difference in areas under the curves (AUC). Corresponding sensitivities and specificities for the full range of possible scores were calculated. Survival analyses were conducted on an expanded sample of MCI patients (n=147) to examine the effect of the UPSIT, BSIT and 10-item scale scores on conversion to AD (mean follow-up was 42 months). This expanded sample included thirteen additional MCI patients who had less than 2 years of follow-up and seven additional MCI patients who had converted to AD after 2 years. The additional twenty MCI patients had either less than 24 months follow up or converted to AD after 24 months.

Results

Demographic and clinical variables and olfactory scores for the controls and MCI and AD patients are summarized in Table 2. Cochran-Armitage linear trend tests revealed that five UPSIT items (cheddar cheese, lime, grass, orange and fruit punch) did not show a decreasing trend across the ordinal groups (Table 3). These five items were excluded from further analyses.

Both LR selection procedures (likelihood score based/AIC and stepwise) yielded the same 10-items (Table 3 bolded items). A list of selection frequencies across 100 bootstrapping samples revealed that the originally selected 10 items were also the most frequently selected across the 100 final item subsets (Table 3). Also, across the final item subsets, regression coefficients consistently indicated that an incorrect response for the 10 items, after adjusting for the other items in the subset, was associated with being classified as an MCI converter or AD patient (Table 3). For the 290 subjects (using the same binary outcome), ROC analyses revealed that the AUC for the 10-item scale was greater than for the UPSIT (AUC: 0.908, S.E.=0.018 vs. 0.882, S.E.=0.020, respectively, p=0.048) and B-SIT (0.841, S.E.=0.023, p<0.001) and the 3-item Pocket Smell Test (PST (lilac, smoke and lemon); 0.717, S.E.=0.029, p<0.0001). A similar AUC pattern was observed when the sample was restricted to 127 MCI subjects, classified as converters within 2 years of follow-up versus non-converters at 2 years (10-item: 0.816, S.E.=0.042; UPSIT: 0.789, S.E.=0.044; B-SIT: 0.756, S.E.=0.050; and the 3-item PST: 0.711, S.E.=0.073, p=0.005). Sensitivities and Specificities for a range of possible scores on each scale are shown in Table 4.

Further, for 290 subjects, AUC (lilac, smoke, lemon)=0.7492, S.E.=0.0273; and AUC (mint, paint thinner, peanut)=0.7661, S.E.=0.0254. The AUC for either one of these mini-tests were significantly smaller than that of the invention's 10-item test, p<0.0001. The pattern remains in the classification of 127 MCI patients for AD conversion within two years that AUC (lilac, smoke, lemon)=0.7151, S.E.=0.0487, p=0.006; AUC (mint, paint thinner, peanut)=0.5813, S.E.=0.0529, p<0.0001.

Age Stratified Cox proportional hazards models for the 147 MCI patients with variable follow-up times showed that scores from the UPSIT and 10-item scale significantly predicted conversion to AD (p<0.000), even after adjusting for gender, education, and MMSE scores (p=0.026 & p<0.00 respectively). The UPSIT and 10-item scale showed maximum risk ratios [RR] of 4.30 (score of ≦32, 95% CI 1.81 to 10.26, p=0.001) and 5.03 (score of ≦7, 95% CI 2.46 to 10.28, p<0.0001) respectively. Controlling for the same covariates reduced the RRs (UPSIT RR: 2.52, 95% CI 0.99 to 6.42, p=0.053; 10-item RR: 3.94, 95% CI 1.79 to 8.67, p<0.001). B-SIT scores adjusted for the same covariates were also associated with conversion to AD (p=0.059; a cutoff score≦8 yielded a maximum RR of 2.21, 95% CI 1.05 to 4.67, p=0.037), as were 3-item PST scores (p=0.003; a cutoff score≦2 yielded a maximum RR of 3.95, 95% CI 1.75 to 8.93, p<0.001). When the sample was restricted to the 127 MCI patients with 2 years of follow-up, age stratified logistic regression analyses that controlled for the same demographic and clinical variables also demonstrated that both the 10-item and 3-item tests predicted conversion to AD (10-item scale: p=0.003; a cutoff score≦7 yielded a maximum odds ration of 5.69, 95% CI 1.92 to 16.08, p=0.0017; PST: p=0.008; a cutoff score≦2 yielded a maximum odds ratio of 5.93, 95% CI 1.99 to 17.67, p=0.0014). Adjusted for the same covariates, score with items mint, paint thinner, and peanut, was not related to AD conversion, while the score with items lilac, smoke, and lemon, was p=0.0027, with cutoff score≦2, RR−3.95, 95% CI 1.75 to 8.93, p=0.0009.

Analysis

Analyses yielded a 10-item scale that performed comparably to the UPSIT with indications of superiority to the B-SIT in classifying subjects with increasing risk of AD. In an expanded sample of MCI patients with variable follow-up times, the 10-item scale and UPSIT predicted conversion to AD after adjusting for clinical and demographic covariates. Importantly, with the data from 290 subjects, two different statistical selection procedures identified the same 10-items. The same 10 items were also the most frequently selected items in 100 bootstrapping samples. Three or more incorrect responses on these 10 items related to risk of AD. These findings suggest that the 10 identified items represent an optimal subset related to risk of AD. The greater risk associated with certain UPSIT items may be explained by AD-related pathology in olfactory-related brain areas that may differentially impact the neural circuitry mediating odor processing.

For olfactory tests to be clinically useful for early detection, they must be sensitive to early pathological and functional changes, significantly add to the predictive accuracy of known demographic and clinical risk factors, and be brief. The proposed 10-item scale meets these criteria, and can be self-administered by patients in 5 minutes with minimal supervision. Scoring is objective and can be performed rapidly by a trained person. The practitioner need only review and interpret the results in the context of a clinical work-up. Pending independent replication, the 10-item scale's potential diagnostic and predictive utility make a strong case for including it as part of a standard clinical workup for patients at risk for AD.

Summary of demographic and clinical variables and smell identification scores for healthy elderly subjects and MCI and AD patients (n=310) are presented in Table 2 below:

TABLE 2
Summary of Variables and Scores

| | Healthy | MCI | AD | | MCI Non- | MCI | |
|---|---|---|---|---|---|---|---|
| | Elderly | Patients | Patients | | converters | Converters | |
| | (n = 63) | (n = 147) | (n = 100) | | (n = 109) | (n = 38) | |
| Demographic | Mean | Mean | Mean | p- | Mean | Mean | p- |
| Variable | (SD) | (SD) | (SD) | value* | (SD) | (SD) | value** |
| Age | 65.71 | 67.43 | 71.72 | <0.001 | 65.59 | 72.71 | <0.001 |
| (years) | (9.38) | (9.85) | (9.54) | | (9.99) | (7.28) | |
| Education | 16.68 | 14.96 | 13.09 | <0.001 | 15.27 | 14.08 | 0.142 |
| (years) | (2.60) | (4.29) | (4.35) | | (4.19) | (4.49) | |
| Sex | 54.0 | 55.1 | 63.8 | 0.33 | 54.1 | 57.9 | .417 |
| (% Female) | | | | | | | |
| Folstein | 29.37 | 27.28 | 19.96 | <0.001 | 27.68 | 26.13 | 0.01 |
| MMSE | (.768) | (3.23) | (5.96) | | 3.43 | (2.21) | |
| UPSIT | 34.86 | 31.22 | 23.72 | <0.001 | 33.02 | 26.05 | <0.001 |
| Score | (4.18) | (6.45) | (6.48) | | (4.68) | (7.96) | |
| B-SIT | 10.60 | 9.56 | 7.04 | <0.001 | 10.12 | 7.95 | <0.001 |
| Score | (1.53) | (2.21) | (2.62) | | (1.70) | (2.67) | |
| 10-item | 8.98 | 8.26 | 5.48 | <0.001 | 8.75 | 6.84 | <0.001 |
| Scale Score | (1.24) | (1.66) | (1.71) | | (1.23) | (1.90) | |

*One-way ANOVA or Fisher's exact test (Sex) were conducted to compare healthy elderly, MCI patients and AD patients.
**t-tests or Fisher's exact test (Sex) were conducted to compare non-converters vs. converters to AD on follow-up evaluation.
MCI: Mild Cognitive Impairment
MMSE: 30-item Mini-Mental State Exam
UPSIT: University of Pennsylvania Smell Identification Test
B-SIT: Brief Smell Identification Test

Percent Correct Smell Identifications across four ordinal groups increasing in risk of AD and corresponding raw and adjusted (multiple test adjusted p-value above 0.20) Cochran-Armitage Linear Trend Test p-values are presented in Table 3 below:

TABLE 3
Percent Correct Smell Identifications

| | | | | | | | | Frequency | Number of |
|---|---|---|---|---|---|---|---|---|---|
| | | | | | | | | (%) in 100 | Negative*** |
| | | | n = 96 | n = 31 | | | | boot- | regression |
| | | | MCI | MCI | | | Multiple | strapping | coefficients |
| | | n = 63 | “Low | “High | n = 100 | | Test | samples | in the 100 |
| UPSIT | Odorant | Healthy | Risk” | Risk” | AD | Raw | Adjusted | (n = 290 | final item |
| Item # | Name | Elderly | Patients | Patients | patients | p-value | p-value** | each) | subsets |
| 1 | Pizza | 91 | 81 | 61 | 61 | <.0001 | <.0001 | 5 | 3 |
| 3* | Menthol | 92 | 85 | 84 | 48 | <.0001 | <.0001 | 62 | 62 |
| 4 | Cherry | 98 | 88 | 81 | 59 | <.0001 | <.0001 | 11 | 10 |
| 5 | Motor Oil | 91 | 90 | 77 | 64 | <.0001 | <.0001 | 31 | 0 |
| 6 | Mint | 95 | 87 | 74 | 55 | <.0001 | <.0001 | 21 | 15 |
| 7 | Banana | 94 | 82 | 68 | 58 | <.0001 | <.0001 | 15 | 1 |
| 8 | Clove | 94 | 94 | 65 | 59 | <.0001 | <.0001 | 88 | 88 |
| 9 | Leather | 97 | 93 | 77 | 50 | <.0001 | <.0001 | 94 | 94 |
| 11 | Onion | 89 | 82 | 68 | 56 | <.0001 | <.0001 | 19 | 19 |
| 13 | Licorice | 97 | 91 | 84 | 61 | <.0001 | <.0001 | 14 | 14 |
| 16 | Gasoline | 95 | 92 | 81 | 67 | <.0001 | <.0001 | 21 | 6 |
| 17 | Strawberry | 92 | 84 | 61 | 56 | <.0001 | <.0001 | 61 | 60 |
| 20 | Gingerbread | 84 | 70 | 55 | 49 | <.0001 | <.0001 | 12 | 8 |
| 21 | Lilac | 94 | 94 | 81 | 62 | <.0001 | <.0001 | 69 | 69 |
| 24 | Root beer | 95 | 91 | 74 | 68 | <.0001 | <.0001 | 34 | 33 |
| [symbol] | [symbol] | [symbol] | [symbol] | [symbol] | [symbol] | [symbol] | [symbol] | [symbol] | [symbol] |
| 29 | Wintergreen | 92 | 83 | 71 | 52 | <.0001 | <.0001 | 13 | 11 |
| 30 | Watermelon | 87 | 85 | 71 | 61 | <.0001 | <.0001 | 31 | 31 |
| 31 | Paint | 95 | 92 | 81 | 60 | <.0001 | <.0001 | 33 | 33 |
| | thinner | | | | | | | | |
| [symbol] | [symbol] | [symbol] | [symbol] | [symbol] | [symbol] | [symbol] | [symbol] | [symbol] | [symbol] |
| 34 | Pine | 92 | 88 | 77 | 58 | <.0001 | <.0001 | 21 | 19 |
| 35 | Grape | 97 | 91 | 68 | 59 | <.0001 | <.0001 | 39 | 39 |
| [symbol] | [symbol] | [symbol] | [symbol] | [symbol] | [symbol] | [symbol] | [symbol] | [symbol] | [symbol] |
| 38 | Natural gas | 87 | 93 | 68 | 58 | <.0001 | <.0001 | 51 | 51 |
| 39 | Rose | 91 | 85 | 61 | 55 | <.0001 | <.0001 | 14 | 6 |
| 40 | Peanut | 98 | 94 | 81 | 68 | <.0001 | <.0001 | 32 | 32 |
| 2 | Bubble gum | 86 | 83 | 55 | 60 | <.0001 | <.0001 | 26 | 26 |
| 25 | Dill pickle | 94 | 76 | 68 | 62 | <.0001 | <.0001 | 42 | 0 |
| 19 | Chocolate | 89 | 85 | 74 | 64 | <.0001 | .0003 | 18 | 16 |
| 23 | Peach | 81 | 84 | 74 | 61 | 0.0002 | .0057 | 7 | 3 |
| 22 | Turpentine | 83 | 80 | 58 | 62 | 0.0003 | .0089 | 9 | 6 |
| [symbol] | [symbol] | [symbol] | [symbol] | [symbol] | [symbol] | [symbol] | [symbol] | [symbol] | [symbol] |
| 18 | Cedar | 84 | 79 | 74 | 63 | 0.0007 | .0192 | 14 | 1 |
| 10 | Coconut | 79 | 79 | 45 | 64 | 0.0024 | .0655 | 5 | 2 |
| 15 | Cinnamon | 83 | 80 | 61 | 67 | 0.0042 | .1068 | 6 | 1 |
| 14 | Cheddar | 64 | 57 | 26 | 53 | 0.0553 | .6938 | 7 | 7 |
| | cheese | | | | | | | | |
| 27 | Lime | 76 | 62 | 58 | 62 | 0.0737 | .7757 | 0 | — |
| 32 | Grass | 67 | 77 | 42 | 64 | 0.0955 | .8473 | 1 | 1 |
| 28 | Orange | 79 | 67 | 61 | 75 | 0.4558 | .9998 | 1 | 0 |
| 12 | Fruit Punch | 43 | 38 | 29 | 56 | 0.9800 | 1.000 | 0 | — |
| *Bolded Odorants comprise the 10-item scale. Italicized and underlined items occur in the B-SIT. The 4 bolded, italicized and underlined items occur in both the B-SIT and the 10-item scale. | | | | | | | | | |
| **Items are ranked according to multiple test adjusted p-values. Note: The bottom 5 items did not show a decreasing trend across groups (P > 0.20). | | | | | | | | | |
| ***Logistic Regression (Beta) coefficients for the UPSIT items in the final selected item subsets indicate the magnitude and direction of the relationship between the given item and the binary outcome (controls plus MCI nonconverters vs. MCI converters plus AD patients) after adjusting for all other | | | | | | | | | |
| # items in the item subset. Negative regression coefficients (frequency are listed in the table for each UPSIT item) indicated that an incorrect response on the given item after adjusting for the other items in the subset is associated with the classification of ‘MCI converter plus AD patient’ and | | | | | | | | | |
| # a correct response with the classification of ‘Control plus MCI nonconverter.’ | | | | | | | | | |

Sensitivity and specificity values for the 40-item UPSIT, 12-item B-SIT and 10-item scale when classifying AD patients and MCI converters within 2 years of follow-up vs. MCI non-converters at two years of follow-up plus controls (n=290) and when restricting the sample to 31 MCI patients who converted to AD within 2 years of follow-up vs. 96 MCI patients who had not converted to AD within 2 years (n=127), are presented in Table 4 below:

TABLE 4
Sensitivity and Specificity Values

| | n = 290 | | n = 127 | |
|---|---|---|---|---|
| | Sensitivity | | Sensitivity | |
| | (≦cutoff | Specificity | (≦cutoff | Specificity |
| | score) | (>cutoff score) | score) | (>cutoff score) |
| UPSIT | | | | |
| Cutoff | | | | |
| Scores | | | | |
| 29 | 77.10 | 84.28 | 58.06 | 80.21 |
| 30 | 82.44 | 81.13 | 61.29 | 77.08 |
| 31 | 87.79 | 76.73 | 70.97 | 73.96 |
| 32 | 91.60 | 69.18 | 83.87 | 62.50 |
| 33 | 93.13 | 64.15 | 87.10 | 56.25 |
| 10-item | | | | |
| Scale | | | | |
| Cutoff Scores | | | | |
| 6 | 63.36 | 93.08 | 45.16 | 92.71 |
| 7 | 83.21 | 89.31 | 58.06 | 87.50 |
| 8 | 94.66 | 67.92 | 83.87 | 65.63 |
| 9 | 97.71 | 38.36 | 96.77 | 34.38 |
| B-SIT | | | | |
| Cutoff | | | | |
| Scores | | | | |
| 7 | 47.33 | 88.68 | 41.94 | 88.54 |
| 8 | 65.65 | 79.25 | 54.84 | 86.46 |
| 9 | 78.63 | 54.72 | 67.74 | 75.00 |
| 10 | 90.08 | 27.04 | 80.65 | 48.96 |
| PST | | | | |
| Cutoff | | | | |
| Score | | | | |
| 3 | 79.39 | 59.75 | 80.65 | 58.33 |

What is claimed:

1. An odor-identification test for determining whether a subject has a neuropsychiatric condition, cognitive disease or disorder, or has a risk for developing a neuropsychiatric condition, cognitive disease or disorder, the test comprising from at least six to about twenty different odorants, wherein the at least six odorants are selected from the group consisting of: menthol, clove, leather, strawberry, lilac, pineapple, smoke, soap, natural gas, and lemon.

2. The test of claim 1, wherein the test comprises from at least seven to about twenty different odorants, wherein at least seven odorants are selected from the group consisting of: menthol, clove, leather, strawberry, lilac, pineapple, smoke, soap, natural gas and lemon, and wherein the additional odorants are selected from the group consisting of: menthol, clove, leather, strawberry, lilac, pineapple, smoke, soap, natural gas, lemon, pizza, cherry, motor oil, mint, banana, onion, licorice, gasoline, gingerbread, root beer, wintergreen, watermelon, paint thinner, pine, grape, rose, peanut, bubble gum, dill pickle, chocolate, peach, turpentine, cedar, coconut, cinnamon, cheddar cheese, lime, grass, orange, and fruit punch.

3. The test of claim 1, wherein the test comprises from at least seven to about twenty different odorants, wherein at least seven odorants are selected from the group consisting of: menthol, clove, leather, strawberry, lilac, pineapple, smoke, soap, natural gas and lemon, and wherein the additional odorants are selected from the group consisting of: menthol, clove, leather, strawberry, lilac, pineapple, smoke, soap, natural gas, lemon, pizza, cherry, motor oil, mint, banana, onion, licorice, gasoline, gingerbread, root beer, wintergreen, watermelon, paint thinner, pine, grape, rose, peanut, bubble gum, dill pickle, chocolate, peach, turpentine, cedar, coconut, and cinnamon.

4. An odor-identification test for determining whether a subject has a neuropsychiatric condition, cognitive disease or disorder, or a risk for developing a neuropsychiatric condition, cognitive disease or disorder, the test comprising ten different odorants, wherein the ten odorants are menthol, clove, leather, strawberry, lilac, pineapple, smoke, soap, natural gas, and lemon.

5. The test of claim 1, 2, 3, or 4, wherein the odorants are contained in microcapsules, an absorbent substance, or in a bottle.

6. The test of claim 5, wherein the microcapsules are embedded in a strip.

7. The test of claim 1, 2, 3, or 4, wherein the test further comprises a multiple-choice question matched to each odorant, wherein each multiple-choice question asks a test-taker to identify the name of the matched odorant from a set of odorant names.

8. The test of claim 1, 2, 3, or 4, wherein the test further comprises a booklet, wherein the booklet comprises a different page for each odorant, wherein each page comprises: (a) a strip comprising microcapsules comprising an odorant, and (b) a multiple-choice question asking a test-taker to identify the name of the odorant.

9. The test of claim 1, 2, 3, or 4, wherein the neuropsychiatric condition, cognitive disease or disorder comprises Alzheimer's disease, Parkinson's disease, Huntington's disease, Korsakoff's amnestic syndrome, acquired immunodeficiency syndrome (AIDS), amyotrophic lateral sclerosis, motor neuron disease, schizophrenia, advanced anorexia, frontotemporal dementia, lewy body dementia, vascular dementia, or a combination thereof.

10. The test of claim 1, 2, 3, or 4, wherein the neuropsychiatric condition, cognitive disease or disorder comprises Alzheimer's disease.

11. A method for discriminating whether a subject is normal or suffers from a neuropsychiatric condition, cognitive disease or disorder, the method comprising:
(a) administering to the subject an odor-identification test according to claim 7;
(b) scoring the number of odorants correctly identified by the subject; and
(c) analyzing the score to diagnose the subject as normal or as having a neuropsychiatric condition, cognitive disease or disorder.

12. The method of claim 11, wherein the subject is diagnosed as normal if the subject correctly identified at least 70% of the odorants.

13. The method of claim 11, wherein the subject is diagnosed as normal if the subject correctly identified at least 80% of the odorants.

14. The method of claim 11, wherein the neuropsychiatric condition, cognitive disease or disorder comprises Alzheimer's disease, Parkinson's disease, Huntington's disease, Korsakoff's amnestic syndrome, acquired immunodeficiency syndrome (AIDS), amyotrophic lateral sclerosis, motor neuron disease, schizophrenia, or advanced anorexia.

15. The method of claim 11, wherein the neuropsychiatric condition, cognitive disease or disorder comprises Alzheimer's disease.

16. A method for predicting whether a subject who suffers from a mild cognitive disorder will develop a more severe neuropsychiatric condition, cognitive disease or disorder, the method comprising:
(a) administering to the subject an odor-identification test according to claim 7;
(b) scoring the number of odorants correctly identified by the subject; and
(c) analyzing the score to predict whether the subject will develop a more severe neuropsychiatric condition, cognitive disease or disorder.

17. The method of claim 16, wherein the subject is not predicted to develop a more severe neuropsychiatric condition, cognitive disease or disorder if the subject correctly identified at least 70% of the odorants.

18. The method of claim 16, wherein the subject is not predicted to develop a more severe neuropsychiatric condition, cognitive disease or disorder if the subject correctly identified at least 80% of the odorants.

19. The method of claim 16, wherein the mild cognitive disorder comprises Mild Cognitive Impairment (MCI), age associated memory impairment (AAMI), age related cognitive decline (ARCD), Benign Senescent Forgetfulness (BSF), or Cognitive Impairment No Dementia (CIND); and wherein the neuropsychiatric condition, cognitive disease or disorder comprises Alzheimer's disease, Parkinson's disease, Huntington's disease, Korsakoff's amnestic syndrome, acquired immunodeficiency syndrome (AIDS), amyotrophic lateral sclerosis, motor neuron disease, schizophrenia, or advanced anorexia.

20. The method of claim 16, wherein the neuropsychiatric condition, cognitive disease or disorder comprises Alzheimer's disease, and wherein the mild cognitive disorder comprises Mild Cognitive Impairment.

21. A odor-identification test kit for determining whether a subject has Alzheimer's disease, or a risk for developing Alzheimer's disease, the odor-identification test kit comprising:
(a) an odor-identification test according to claim 4; and
(b) instructions for taking the test.

22. The odor-identification test kit of claim 21, wherein the odorants are contained in microcapsules, wherein the microcapsules for each odorant are embedded in a strip, wherein there is a different strip for the microcapsules for each odorant.

23. The odor-identification test kit of claim 22, wherein the test further comprises a multiple-choice question matched to each strip, and wherein the instructions comprise:
(a) an instruction to a test-taker to scratch the strip with a pencil or another pointed device;
(b) an instruction to the test-taker to smell the scratched strip; and
(c) an instruction to the test-taker to answer the multiple-choice question matched to the strip, wherein the multiple-choice question asks the test-taker to identify the name of the odorant from the scratched strip, and wherein the multiple-choice question provides a set of potential answers comprising different odorant names.

24. The odor-identification test kit of claim 23, further comprising an answer-key.

25. An odor-identification test for determining whether a subject has a neuropsychiatric condition, cognitive disease or disorder, or has a risk for developing a neuropsychiatric condition, cognitive disease or disorder, the test consisting of about 10 items.

26. An odor-identification test for determining whether a subject has a neuropsychiatric condition, cognitive disease or disorder, or has a risk for developing a neuropsychiatric condition, cognitive disease or disorder, wherein the items have an intense odor, and comprising from at least six to about twenty different odorants, wherein the at least six odorants are selected from the group consisting of: lemon, paint thinner, dill pickle, smoke, onion, leather, turpentine, gasoline, peanut, and coconut.
